(19) United States
(12) Patent Application Publication
(10) Pub. No.: US 2025/0255487 A1
(43) Pub. Date: Aug. 14, 2025

(54) SYSTEMS AND METHODS FOR ORAL INSPECTION

(71) Applicant: Dyson Technology Limited, Malmesbury Wiltshire (GB)

(72) Inventors: Michele BENETTI, Bristol (GB); Bartosz SLAK, Chippenham (GB); Christopher John BATEMAN, Swindon (GB)

(21) Appl. No.: 18/859,699

(22) PCT Filed: Apr. 25, 2023
(86) PCT No.: PCT/GB2023/051088

§ 371 Date: Oct. 24, 2024

(30) Foreign Application Priority Data
Apr. 29, 2022 (GB) 2206321.8

(51) Int. Cl. A61B 5/00

(52) U.S. Cl. CPC A61B 5/0088; A61B 5/0071; A61B 5/6898

(57) ABSTRACT

A dentine-enamel boundary detection system for an oral inspection device is provided. The dentine-enamel boundary detection system includes a light emission module configured to emit light to irradiate an oral region of interest and two optical filters having different respective passbands. Each of the two optical filters are arranged to filter a portion of fluorescence emitted from oral structures in the oral region of interest to pass filtered fluorescence. The system further includes a sensor module and a processor module. The sensor module is configured to detect the filtered fluorescence and output corresponding sensor data. The processor module configured to identify the presence of a dentine-enamel boundary in the oral region of interest by processing the sensor data output by the sensor module.

### FIELD OF THE INVENTION

The present invention relates generally to devices and methods for oral inspection, and in particular, for inspection of oral structures. In particular, but not exclusively. the present disclosure concerns dentine-enamel boundary systems for detecting the dentine-enamel boundary of teeth, which may be used to identify gaps between teeth and/or assess oral health, as well as to devices incorporating such systems, and uses of such systems.

### BACKGROUND

Various systems comprising a camera for oral inspection are known (sometimes referred to as ‘dental intra-oral camera systems’). These devices are generally used for imaging of the teeth, gums, or other oral structures of a patient in a medical setting-for example to identify one or more oral structures such as teeth, or to distinguish between abnormal and healthy tooth surfaces by identifying plaques or caries on or within teeth, to determine whether one or more treatments should be applied to the oral structures.

However, there are a number of problems to be overcome with respect to use of conventional imaging systems for use in oral inspection. One primary issue is that in the environment of a user's mouth, and in particular during a dental cleaning process, multiple obscuration mediums may be present: for example, water, saliva, toothpaste and foam. Accordingly, when such obscurants are present, it may be more difficult to accurately identify, or distinguish between, different oral structures in a user's mouth.

Indeed, it may be particularly desirable to be able to detect an interdental gap between a user's teeth, for example for the purpose of observing a health state of the teeth adjacent the interdental gap location, or for applying a treatment to the interdental gap. In view of the above problem relating to visual identification of an interdental gap due to obscurants which may be present in an oral environment, interdental detection systems typically require physical probing of, or direct contact with, a user's teeth in order to identify an interdental gap.

For example, EP3888589A1 discloses an interdental space detection component for use with an interdental care device, which includes an interdental probe insertable into an oral cavity of a user for interdental space detection. In this example, optical detectors may be used to determine the intensity of light on either side of a probe, and hence derive an intensity profile with distance from the tooth. That is, the probe is required to be in close contact with teeth to provide an indication of whether the probe is in an interdental gap, and a time series of data is required to determine a probe position.

It would be desirable to provide a solution for identifying an interdental gap which does not require physical interaction with a user's teeth, can provide information at a given time point, and that is also able to satisfactorily identify the gap even in the presence of one or more obscuration mediums.

The present invention has been devised in light of the above considerations.

### SUMMARY OF THE INVENTION

According to a first aspect of the present invention, there is provided a dentine-enamel boundary detection system for an oral inspection device, the dentine-enamel boundary detection system comprising: a light emission module configured to emit light to irradiate an oral region of interest: two optical filters having different respective passbands, each of the two filters arranged to filter a portion of fluorescence emitted from oral structures (e.g. teeth) in the oral region of interest to pass filtered fluorescence; and a sensor module configured to detect the filtered fluorescence and output corresponding sensor data: wherein the system further comprises a processor module configured to identify the presence of a dentine-enamel boundary in the oral region of interest by processing the sensor data output by the sensor module.

The term “oral structures” is used to refer to physical structures present in an oral cavity, for example teeth.

An optical filter is a device that preferentially transmits light of at least some wavelengths, whilst reducing or preventing transmission of light of at least some other wavelengths. The term “preferentially filters” is used herein to define that the filter is configured to preferentially absorb light within a set or range of predetermined wavelengths. Dentine and enamel each have different fluorescence emission spectra, and so by providing two filters with different passbands which are each arranged to filter a portion of fluorescence which is emitted from oral structures, data received at the sensor module can be used to identify the presence of a dentine-enamel boundary. As fluorescence light is relied upon to identify the dentine-enamel boundary, this boundary may be identified even in the presence of one or more obscuration mediums. Properties of the dentine-enamel boundary may be used to assess oral health. For example, a size of the boundary (e.g. a width) may be used to assess the extent of enamel damage, wherein a larger (e.g. wider) boundary area may indicate an increased level of enamel erosion or damage compared with a smaller (e.g. narrower) boundary. Furthermore, as enamel erosion principally occurs at the edges of teeth to expose the underlying dentine at these regions the system may also be used to identify gaps between teeth.

As noted above, the light module is configured to emit light to irradiate an oral region of interest. The light emission module may be configured to emit light having a wavelength in a visible part of the electromagnetic spectrum, and/or light in a non-visible part of the electromagnetic spectrum.

Optionally, the light emission module is configured to emit light having a wavelength in a range of 405 to 450 nm. That is, the light emission module is configured to emit light in a near-UV range. In this way. the light emission module is configured to ensure strong fluorescence of both dentine and enamel in the oral region of interest. Preferably, the light emission module does not emit light having a wavelength in a UV range (i.e. at wavelengths of 400 nm or less), and the near-UV range is used such that the health risk to a user of the device may be reduced. Accordingly, the light emission module may be configured to emit light having a wavelength of 405 nm or more. 410 nm or more. 415 nm or more, or 420 nm or more. However, it may be desirable for the emission wavelength not to be too high. This is because light of higher wavelengths may not have sufficient energy to excite fluorescence of teeth. Accordingly in some embodiments, the light emission module may be configured to emit light having a wavelength of 450 nm or less, 440 nm or less, or 430 nm or less. In some preferred embodiments, the light emission module may be configured to emit light having a wavelength in a range of from 405 to 420 nm.

The specific form of the light emission module is not particularly limited. In some embodiments, the light emission module may comprise one or more light emitting diodes, LEDs. for example. In other embodiments, the light emission module may comprise a laser light source. In some embodiments, the light emission module may be located near, or adjacent to, the sensor module.

The specific form of the optical filters used in the present invention is not particularly limited, other than they must be suitable for being arranged in an optical path between the oral region of interest and the sensor module In some embodiments, the optical filters may be bulk-dyed filters. In other embodiments the optical filters may comprise an interference coating. In some embodiments the filters may each comprise an absorptive filter, an interference filter, or a dichroic filter. Preferably the filters comprise an absorptive filter, as absorptive filters are generally relatively low cost and readily available. The optical filters may be made from any suitable material: for example, they may be made from a glass material or a polymeric material (such as a resin). In some examples. the two optical filters may be or form part of a Bayer filter arrangement which is part of or placed over the sensor module. For example, the sensor module may comprise a pixel array, and the two optical filters may form part of a Bayer arrangement over the pixel array of the sensor module By being arrangement in this way, different channels of data relating to fluorescence which is filtered by each of the optical filters may be read directly from the sensor module itself.

Optionally, the two optical filters include a blue filter configured to preferentially pass filtered fluorescence having a wavelength of between 430 nm and 560 nm and a red filter configured to preferentially pass filtered fluorescence having a wavelength of 560 nm or more. This may be achieved by selecting appropriate colours for the filters. in particular where the filter is a bulk-dyed filter. The present inventors have realised that by two optical filters having passbands which are configured in this way. the filtered fluorescence which is detected by the sensor module is particularly indicative of differing levels of dentine and enamel in the oral region of interest highlighted as a result of a difference in the fluorescence spectra of dentine and enamel. In particular, it has been found that dentine and enamel each fluoresce strongly in the passband of the blue filter, whereas the fluorescence spectrum of enamel has a higher intensity than dentine in the region above 560 nm. Therefore, by comparing the filtered fluorescence in these passband regions the dentine-enamel boundary may be readily identified by processes described in more detail below.

The precise form of the sensor module is not particularly limited, provided that it is configured to detect light reflected from or emitted by the oral region of interest and output corresponding sensor data. By detecting and analysing sensor module data, the detection system may identify the presence of a dentine-enamel boundary in the oral region of interest at a particular time (e.g. when an image is taken). The sensor module may comprise a number of pixels (or picture elements), and so the sensor module may be referred to herein as a ‘pixelated’ sensor module, in some examples. In some embodiments, the sensor module comprises a camera (e.g., a large 2D array of pixels or sensors) or light detector. The sensor data output by the sensor module will typically comprise image data generated by the sensor module. Accordingly, the phrases “output sensor data” and “generated image data” are used interchangeably in the following disclosure. In some embodiments, the generated image data (output sensor data) comprises red, green and blue (RGB) image data (for example, corresponding to respective RGB pixels). The use of RGB image data may allow for more accurate gap localisation compared to other types of image data. Other types of image data (e.g. black and white image data) may be used in alternative embodiments.

Optionally, the sensor module may be configured to detect a time of arrival at the sensor module of the filtered fluorescence and output corresponding time data, and the processor module may be configured to process the time data output by the sensor module to identify the presence of a dentine-enamel boundary in the oral region of interest. The present inventors have realised that the fluorescence lifetime of dentine differs from the fluorescence lifetime of enamel. In particular, it has been found that the fluorescence lifetime of dentine is longer than the fluorescence lifetime of enamel. By detecting the time of arrival of light which is filtered by each of the two optical filters a dentine-enamel boundary may therefore be identified by processes described in more detail below. For example, the sensor module may comprise a single photon avalanche diode (SPAD). for example forming part of a 2D array of SPADs wherein each SPAD provides a pixel in a SPAD image sensor, which can be used for timing the arrival of photons which pass through each of the two optical filters. Alternatively, the sensor module may comprise a gated imaging device. utilising a complementary metal-oxide-semiconductor (CMOS) sensor. for example. Of course, it will be appreciated that any suitable time-of-flight camera arrangement may be used in embodiments of the invention. In some examples, the light emission module may be configured to produce a repetition of short pulses of light, and the sensor module may be configured to detect a time of arrival at the sensor module of the filtered fluorescence emission and output information related to the delay of the fluorescence with respect the time of each pulse.

Optionally, the dentine-enamel boundary detection system may further comprise two polarisation filters having orthogonal polarisations, each of the two polarisation filters being arranged to filter a portion of the fluorescence emitted from oral structures in the oral region of interest to pass polarised fluorescence. The present inventors have realised that light emitted, reflected, and/or scattered from each of dentine, enamel, and the dentine-enamel boundary has different polarization properties. As a result, light which passes through the polarization filters to the sensor module carries information relating to each of these features, such that the presence of a dentine-enamel boundary can be detected on the basis of the sensor output data related to the polarization state of light according to processes described in more detail below. Optionally, the light emission module may be configured to emit polarised light, which may further aid the identification of the dentine-enamel boundary using polarisation filters.

Optionally, the processor module may be configured to identify the presence of an interdental gap in the oral region of interest. That is, the processor module may utilise the identification of a dentine-enamel boundary region in the oral region of interest in order to identify the presence of an interdental gap, which may be particularly advantageous for ensuring that any treatments are preferentially applied to an interdental gap, which can be important for ensuring good oral health as described in more detail below.

According to a second aspect of the present invention. there is provided an oral inspection or treatment device incorporating the dentine-enamel boundary detection system according to the first aspect.

Optionally, the oral inspection or treatment device may be a dental cleaning appliance, such as a toothbrush, further comprising a body and a cleaning tool head.

Optionally, one or both of the light emission module and the sensor module of the dentine-enamel boundary detection system may be provided on the cleaning tool head. This may help allow the dentine-enamel boundary detection system to operate while the dental cleaning appliance is in use, and may provide for more accurate detection of the dentine-enamel boundary by positioning one or both of the light emission module and the sensor module in the vicinity of the oral region of interest. For example, positioning the light emission module in this way may ensure that the light which illuminates the oral region of interest has a high intensity to provide a strong fluorescence response. Similarly, positioning the sensor module in this way may help ensure accurate identification of the dentine-enamel boundary. Alternatively or additionally, the light emission module and/or the sensor module may be at least partially comprised in the body of the device. By arranging the image sensor equipment at least partially in the body of the device, on-device space may be managed more efficiently. That is, the head of the device may be relatively small compared to the body. Further, in embodiments, the head of the device may be separable from the body. It may also be disposable, and it may be desired for a user to replace the head periodically after use. Arranging the light emission module and/or the sensor module at least partially in the body, as opposed to entirely in the head, may thus reduce the cost of replacement parts. Where the light emission module and/or the sensor module may be at least partially comprised in the body of the device. one or more guide components may be provided to conduct light from/to the light emission module and/or the sensor module. The guide components may comprise one or more fibre optic cables.

Optionally, the processor module may be provided as part of a controller module located within a remote device, wherein the remote device and the oral inspection or treatment device may be configured for communication with one another to allow for exchange of signals and/or data. The controller module may be operable to perform various data processing and/or control functions by means of one or more processors. The controller and/or processor forming part of the controller module may additionally be operable to perform various other data processing and/or control functions in addition to processing of the sensor data output by the sensor module to identify the presence of an interdental gap in the oral region of interest Operations performed by the processor module may be carried out by hardware and/or software. Providing the processor module within a remote device may help to ensure that the oral inspection or treatment device can meet any limitations on its size (for example, it may be required to be a handheld device), and also increase efficiency of the system. For example. by providing a remote processor module in this way, the oral inspection or treatment device may require less power and/or computing resources. Optionally, on identification of the presence of an interdental gap in the oral region of interest, the dental cleaning appliance may be configured to apply a treatment to the identified interdental gap in the oral region of interest. Dental plaque can particularly build up in interdental gaps, and so identification and application of treatments to this region can help to ensure improve oral health.

According to a third aspect of the present invention, there is provided a method for detecting a dentine-enamel boundary in an oral region of interest, the method comprising steps of: irradiating an oral region of interest with light; detecting light reflected from or emitted by the oral region of interest, said light comprising filtered fluorescence. the filtered fluorescence having passed through two optical filters having different respective passbands, each of the two filters arranged to filter a portion of fluorescence emitted from oral structures in the oral region of interest to pass said filtered fluorescence; outputting sensor data corresponding to the detected filtered fluorescence; and processing said sensor data to identify the presence of a dentine-enamel boundary in the oral region of interest based on the filtered fluorescence. As explained above, dentine and enamel each have different fluorescence emission spectra, and so by passing fluorescence light emitted from oral structures through two filters with different passbands, data received from a sensor arranged to detect the filter fluorescence can be used to identify the presence of a dentine-enamel boundary. As fluorescence light is relied upon to identify the dentine-enamel boundary, this boundary may be identified even in the presence of one or more obscuration mediums. Properties of the dentine-enamel boundary may be used to assess oral health. For example, a size of the boundary (e.g. a width) may be used to assess the extent of enamel damage, wherein a larger (e.g. wider) boundary area may indicate an increased level of enamel erosion or damage compared with a smaller (e.g. narrower) boundary. Furthermore, as enamel erosion principally occurs at the edges of teeth to expose the underlying dentine at these regions the method may also be used to identify gaps between teeth.

Optionally, processing said sensor data to identify the presence of a dentine-enamel boundary in the oral region of interest may comprise creating a ratiometric image of the oral region of interest based on the ratio of intensity of filtered fluorescence passed through the two optical filters. As described above, the present inventors have realised that by using two optical filters which have different passbands, the filtered fluorescence which is detected by the sensor is particularly indicative of differing levels of dentine and enamel in the oral region of interest highlighted as a result of a difference in the fluorescence spectra of dentine and enamel. The ratiometric image may be created by taking the ratio of the intensity of filtered fluorescence passed through a first of the two optical filters to the intensity of filtered fluorescence passed through a second of the two optical filters for each pixel position. For example, the two optical filters may form part of a Bayer filter which overlies a pixel array of an image sensor Creating a ratiometric image may comprise taking a ratio between two channels (e.g. a ratio between a red and a blue channel) read out from the image sensor. As a result of a difference in the fluorescence spectra of dentine and enamel, the ratiometric image which is created in this way makes the dentine-enamel boundary clear and more easily identifiable.

Optionally, the light irradiating the oral region of interest may have a wavelength in a range of 405 to 450 nm. That is, the light irradiating the oral region of interest may be in a near-UV range. In this way, the light is configured to ensure strong fluorescence of both dentine and enamel in the oral region of interest. Preferably, the light irradiating the oral region of interest may not have a wavelength in a UV range (i.e. at wavelengths of 400 nm or less). and the near-UV range is used such that the health risk to a user may be reduced. Accordingly, the light irradiating the oral region of interest may have a wavelength of 405 nm or more, 410 nm or more, 415 nm or more, or 420 nm or more. However, it may be desirable for the wavelength not to be too high. This is because light of higher wavelengths may not have sufficient energy to excite fluorescence of teeth. Accordingly in some embodiments, the light irradiating the oral region of interest may have a wavelength of 450 nm or less, 440 nm or less, or 430 nm or less. In some preferred embodiments, the light irradiating the oral region of interest may have a wavelength in a range of from 405 to 420 nm.

Optionally, outputting sensor data corresponding to the detected light may comprise outputting time data identifying the time of arrival at the sensor of said light, and processing said sensor data to identify the presence of a dentine-enamel boundary in the oral region of interest may comprise processing the time data. The present inventors have realised that the fluorescence lifetime of dentine differs from the fluorescence lifetime of enamel. In particular, it has been found that the fluorescence lifetime of dentine is longer than the fluorescence lifetime of enamel. By detecting the time of arrival of light which is filtered by each of the two optical filters a dentine-enamel boundary may therefore be identified.

In certain embodiments, processing said sensor data to identify the presence of a dentine-enamel boundary in the oral region of interest may comprise creating a fluorescence lifetime image based on the time data. For example, the intensity and/or colour of each pixel in the fluorescence lifetime image may be determined by a fluorescence lifetime. based on the time of arrival of light which is filtered by each of the two optical filters. The resulting image which is created in this way makes the dentine-enamel boundary clear, as a result of the different fluorescence lifetimes of dentine and enamel, and more easily identifiable.

Optionally, detecting light reflected from or emitted by the oral region of interest may comprise detecting polarised fluorescence, the polarised fluorescence having passed through two polarisation filters having orthogonal polarisations, each of the two polarisation filters arranged to filter a portion of fluorescence emitted from oral structures in the oral region of interest to pass said polarised fluorescence. The present inventors have realised that light emitted, reflected, and/or scattered from each of dentine, enamel, and the dentine-enamel boundary has different polarization properties. As a result, light which passes through the polarization filters carries information relating to each of these features, such that the presence of a dentine-enamel boundary can be detected on the basis of the polarization state of light. Optionally, in certain embodiments, irradiating an oral region of interest with light may comprise irradiating an oral region of interest with polarised light, which may further aid the identification of the dentine-enamel boundary using polarisation filters.

In some embodiments, processing said sensor data to identify the presence of a dentine-enamel boundary in the oral region of interest may comprise calculating the Stokes parameters from the orthogonal polarization directions of the polarised fluorescence. Additionally, and/or alternatively, processing said sensor data to identify the presence of a dentine-enamel boundary in the oral region of interest may comprise creating a ratiometric image of the oral region of interest based on the ratio of intensity of polarised fluorescence passed through the two polarisation filters. For example, the ratiometric image may be created by taking the ratio of the intensity of filtered fluorescence passed through a first of the two polarisation filters to the intensity of filtered fluorescence passed through a second of the two polarisation filters for each pixel position. Creating a ratiometric image may comprise taking a ratio between two channels read out from an image sensor, where the image may have an overlying grid of polarisation filters. As a result of a difference in the polarisation spectra of fluorescence from dentine and enamel, the ratiometric image which is created in this way makes the dentine-enamel boundary clear and more easily identifiable.

A method according to the third aspect of the present invention may be performed by an enamel-dentine boundary detection system according to the first aspect of the present invention, or by an oral inspection or treatment device according to the second aspect of the invention, for example.

The invention includes the combination of the aspects and preferred features described except where such a combination is clearly impermissible or expressly avoided. For example, a method of the invention may incorporate any of the features described with reference to an apparatus of the invention and vice versa.

### SUMMARY OF THE FIGURES

Embodiments and experiments illustrating the principles of the invention will now be discussed with reference to the accompanying figures in which:

FIG. 1 is a schematic drawing of a dentine-enamel boundary detection system according to an embodiment of the present invention;

FIG. 2 is a schematic drawing of a filter arrangement and an image sensor which may be used in embodiments of the present invention;

FIG. 3 is a schematic drawing of an oral treatment device according to an embodiment of the present invention;

FIG. 4 is a flow diagram showing a method of operating an oral treatment device according to an embodiment of the present invention; and

FIG. 5(a) shows a first channel of an image of oral structures, FIG. 5(b) shows a second channel of an image of oral structures, and FIG. 5(c) shows a ratiometric image derived from FIGS. 5(a) and 5(b).

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

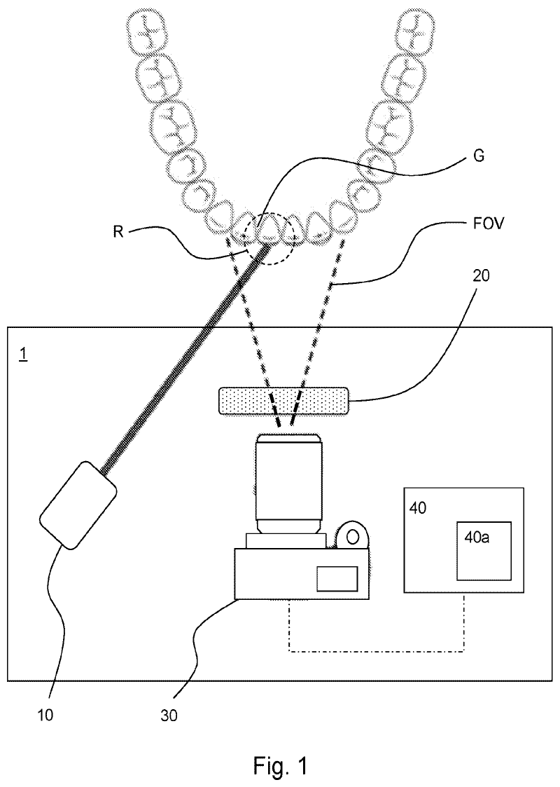

FIG. 1. is a schematic drawing of a dentine-enamel boundary detection system 1 according to an embodiment of the present invention, arranged to identify the presence of a dentine-enamel boundary and an interdental gap G within an oral region of interest R in the oral cavity of a user. In the schematic drawing shown, it can be seen that components of the system are not incorporated in a single device, but rather, are provided as a plurality of separate components. The system includes a light emission module 10, an optical filter arrangement 20, a sensor module 30, and a controller 40 including a processor module 40a.

The light emission module 10 is here conveniently provided as a laser which is configured to emit light in a ‘near-UV’ range, at a wavelength of 405 nm. The laser is arranged to irradiate an oral region of interest R within an oral cavity. Here the oral region of interest includes various oral structures including one or more teeth. Irradiating the oral region of interest with light having a wavelength of 405 nm allows for fluorescence of teeth which are present within the oral region of interest. However, because the light has a wavelength which lies in the ‘near-UV’ region of the electromagnetic spectrum, rather than a wavelength that lies in a UV range of the electromagnetic spectrum, the health risk to a user of the device may be reduced. In some preferred embodiments, the wavelength of light emitted by the light emission module is greater than 405 nm, for example 410 nm or more—this may further reduce any health risk to the user.

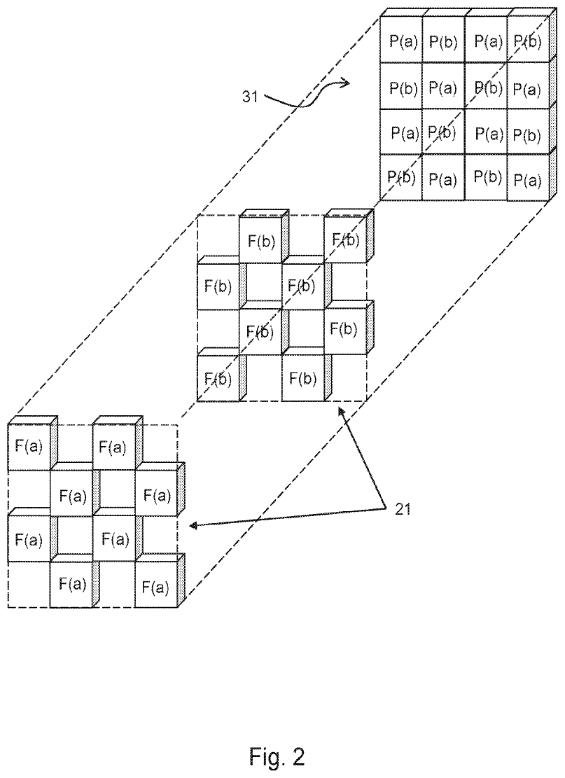

The optical filter arrangement 20 in this embodiment comprises two optical filters having different respective passbands. For example, the two optical filters may be positioned adjacent to one another. In some embodiments the optical filter arrangement 20 may also include two polarisation filters having orthogonal polarisations. The optical filter arrangement 20 may, in some examples, comprise a mosaiced array of different filtering elements, as described in more detail below with respect to FIG. 2, which overlie a pixel array of an image sensor. For example, the array of filtering elements may comprise optical filters with different passbands, and. optionally, polarisation filters. The optical filter arrangement 20 is arranged to lie in an optical path between the oral region of interest and the sensor module (as indicated by the dashed lines showing the field of view (FOV) of the sensor module).

A first of the two optical filters will preferentially transmit light having a wavelength of between 430 nm and 560 nm. and will preferentially filter or block light having wavelengths which fall outside this range. A second of the two optical filters will preferentially transmit light having a wavelength of 560 nm or more, and will preferentially filter or block light having wavelengths shorter than this. This means that the first optical filter will pass fluorescence emitted by both dentine and enamel in the oral region of interest, while the second optical filter will preferentially pass fluorescence emitted by enamel in the oral region of interest (as dentine has very low fluorescence above 560 nm). By comparing data from the sensor module 30 relating to light which is passed through each of the optical filters, a dentine-enamel boundary and an interdental gap may therefore be easily identified. In embodiments where the optical filter arrangement 20 further comprises polarisation filters, a dentine-enamel boundary may also be identified by comparing data from the sensor module 30 relating to light which is passed through each of the polarisation filters.

The sensor module 30 is represented in this schematic figure by a ‘camera’ icon. In practice, the form of the sensor module is not particularly limited, however it preferably comprises one or more image sensors. For example, an image sensor may comprise a 2D array of pixels or picture elements which are sensitive to light, and so the image sensor may be referred to as a pixelated sensor module. Examples of such image sensors include, but are not limited to, charge-coupled devices, CCDs, active-pixel sensors such as complementary metal-oxide-semiconductor, CMOS. sensors, and single photon avalanche diodes, SPADS—for example formed as a 2D array of such elements. Whilst in this embodiment the sensor module is a standalone component arranged to be used outside the oral cavity, in some embodiments, the sensor module is an intraoral sensor—that is, a sensor module operable to be used at least partially inside the oral cavity of a user, in order to generate image data representing the oral cavity of the user. For example, as will be discussed in further detail below, when the dentine-enamel boundary detection system is incorporated in an oral treatment device the sensor module may be at least partially arranged on a head of the oral treatment device which is arranged to be inserted into the oral cavity of the user.

In the present embodiment, as the sensor module 30 is provided as a standalone component, the sensor module comprises one or more processors (not shown). The sensor module is configured to detect light reflected from or emitted by the oral region of interest R within the field of view FOV of the sensor module, said light having passed through the optical filter arrangement 20. The sensor module is configured to output sensor data corresponding to the detected light, which in this case will be generated image data. The generated image data (output sensor data) in this embodiment comprises red, green and blue, RGB, image data.

In some examples, the sensor module 30 may also be configured to detect a time of arrival and the sensor module 30 of the filtered fluorescence and output corresponding time data. For example, the controller 40 may utilise information received from the light emissions module 10 (e.g. time data describing when the oral region of interest R was illuminated) and time data from the sensor module 30 in order to derive information relating to the fluorescence lifetime in order to identify the dentine-enamel boundary.

The sensor module 30 is configured to transmit the output sensor data to the controller 40, as indicated by the dashed line connecting these two components. This transmission may be performed via wires physically connecting the sensor module 30 and the controller 40 or may be by wireless transmission. The controller 40 is operable to perform various data processing and/or control functions, as will be described in more detail below. The controller 40 may comprise one or more components. The one or more components may be implemented in hardware and/or software. The one or more components may be co-located or may be located remotely from each other. The controller 40 may be embodied as one or more software functions and/or hardware modules. The controller 40 comprises at least one processor 40a configured to process instructions and/or data-and in particular. configured to process sensor data which is output from the sensor module 30. Operations performed by the one or more processors 40a may be carried out by hardware and/or software. The controller 40 and/or processor module 40a of the controller are operable to output control signals for controlling one or more components of the dentine-enamel boundary detection system 1, or for controlling an oral treatment device incorporating the dentine-enamel boundary detection system

The processor 40a may be part of a processing system comprising one or more processors and/or memory. The one or more processors of processing systems may comprise a central processing unit (CPU). The one or more processors may comprise a graphics processing unit (GPU). The one or more processors may comprise one or more of a field programmable gate array (FPGA), a programmable logic device (PLD), or a complex programmable logic device (CPLD). The one or more processors may comprise an application specific integrated circuit (ASIC). It will be appreciated by the skilled person that many other types of device, in addition to the examples provided, may be used to provide the one or more processors. The one or more processors may comprise multiple co-located processors or multiple disparately located processors. Operations performed by the one or more processors may be carried out by one or more of hardware, firmware, and software. It will be appreciated that processing systems may comprise more, fewer and/or different components from those described.

In embodiments. the processor 40a processes the sensor data output by the sensor module by means of one or more data analysis algorithm(s). The data analysis algorithm(s) may be configured to analyse received data, e.g. image data, and produce an output useable as a condition by which the dentine-enamel boundary detection system 1, or an oral treatment device incorporating the dentine-enamel boundary detection system 1, may be controlled. For example, they may be configured to output a signal indicative of the presence of a dentine-enamel boundary in the oral region of interest. In embodiments, the data analysis algorithm(s) comprises a classification algorithm, e.g. a non-linear classification algorithm. In embodiments, the data analysis algorithm(s) comprises a trained classification algorithm. However, in alternative embodiments, the data analysis algorithm(s) comprises other types of algorithm, e.g. not necessarily trained and/or not configured to perform classification.

In embodiments, the processor 40a is configured to process the generated image data using a sliding window, which allows for location data of a dentine-enamel boundary to be determined by detecting the presence of the dentine-enamel boundary within the sliding window, and determining a sub-region of the image which contains the boundary. In a sliding window image analysis technique, the sliding window passes across the image, defining sub-regions of the image, and a determination is made on whether a boundary exists in each sub-region of the image. This is described in more detail below.

In embodiments, the generated image data is processed by extracting one or more image features from the image data, and using the extracted one or more image features to determine the location data. The image features may comprise texture-based image features, for example. Since an image consists of pixels which are highly related to each other, image feature extraction is used to obtain the most representative and informative (i.e. non-redundant) information of an image, in order to reduce dimensionality and/or facilitate learning of the classification algorithm.

In embodiments, the one or more image features are extracted using a discrete wavelet transform. The discrete wavelet transform can capture both frequency and location information in an image. For example, for detecting an interdental gap based on images used to identify the dentine-enamel boundary, the image frequency in gap areas is typically higher than the image frequency in teeth or gum areas. This allows a discrete wavelet transform to produce a frequency map of the image that is usable to detect interproximal gaps. In embodiments, a Haar wavelet is used, which has a relatively low computational complexity and low memory usage compared to other wavelets. The coefficients of the wavelet transform (or approximations thereof) may be used as the extracted image features. For example, the output of feature extraction based on the Haar wavelet applied to an image of size a×a may include a horizontal wave h (a/4×a/4), a vertical wave v (a/4×a/4) and a diagonal wave d (a/4×a/4). Other wavelets can be used in alternative embodiments.

The extracted features may be used for a sliding window applied to the image. For example, in the image sub-region defined by the sliding window, a 2×2 pooling for each of h, v and d may be performed, before h, v and d are vectorised and combined into one vector with size 1×108. This may be normalised, along with trained data from the trained classification algorithm, e.g. trained mean and variance values. A support-vector machine, SVM, may be used as a non-probabilistic non-linear binary classifier with a Gaussian radial basis function kernel, which receives the normalised data from the previous step. The trained SVM comprises support vectors having trained coefficients and biases, i.e. determined during a previous training phase. For example, given a set of images together with ground truth labelling, the classification algorithm can be trained so as to assign new examples to one category (e.g. dentine-enamel boundary region) or another (e.g. non-boundary).

In embodiments the one or more image features are extracted using at least one of: an edge detector, a corner detector, and a blob extractor. Extracting image features using such methods may provide a more accurate detection and/or localisation of the dentine-enamel boundary compared to other methods.

In embodiments, the generated image data is processed using a trained classification algorithm configured to detect a dentine-enamel boundary. Using such a trained algorithm results in a more accurate and/or reliable boundary detection compared to a case in which a trained algorithm is not used. In embodiments, the classification algorithm comprises a machine learning algorithm. Such a machine learning algorithm may improve (e.g. increase accuracy and/or reliability of classification) through experience and/or training.

In embodiments, the generated image data is processed to determine at least one characteristic of the identified dentine-enamel boundary. In embodiments, the at least one characteristic is determined by processing the generated image data using a machine learning algorithm. The machine learning algorithm is trained to identify information for use in distinguishing between boundaries. Such information comprises features that are representative of the boundary, i.e. non-redundant features, and/or features which are predicted to vary between boundaries. The identified information may comprise the at least one characteristic of the boundary. In embodiments, such a machine learning algorithm (or one or more different machine learning algorithms) is also used to determine the at least one characteristic of the one or more previously identified boundaries. e.g. features that are representative of the previously identified boundaries and/or useable to distinguish between boundaries, and which are used to compare the previously identified boundaries with the currently identified boundary. In alternative embodiments, characteristic features of the boundaries are extracted from raw image data without the use of machine learning algorithms.

In embodiments, the classification algorithm is modified using the output signals and/or the generated image data. That is, the classification algorithm may be trained and/or further trained using the generated signals and/or the generated image data. Modifying the classification algorithm allows the accuracy and/or reliability of the algorithm to improve through experience and/or using more training data. That is, a confidence level of the determined dentine-enamel boundary location may be increased. Further, modifying the classification algorithm allows the classification algorithm to be tailored to the user. By using the generated signals and/or the generated image data as training data to dynamically re-train the classification algorithm, the classification algorithm can more reliably determine the intraoral location of a dentine-enamel boundary.

In embodiments. training data is received from a remote device. The training data may be received from a network, e.g. “the Cloud’. Such training data may comprise classification data associated with other users. Such training data may comprise crowd-sourced data, for example. In embodiments, such training data may be greater in volume than classification data obtained using the dentine-enamel boundary detection system directly. The use of the training data from the remote device to modify the classification algorithm can increase the accuracy and/or reliability of the classification algorithm compared to a case in which such training data is not used.

FIG. 2 is a schematic drawing of a filter arrangement 21 and an image sensor 31 which may be used in embodiments of the present invention. For example, the filter arrangement may be used in the dentine-enamel boundary detection system 1 described with respect to FIG. 1.

The image sensor 31 comprises an array of pixels P(a), P(b). For example, the image sensor 31 may be a CCD, such that the pixels P(a). P(b) are an array of linked capacitors which are sensitive to incident light. In the arrangement of FIG. 2, the filter arrangement 21 comprises a mosaic formed of two types of filters F(a), F(b). For example, the two types of filters may be optical filters, e.g. a blue filter type configured to preferentially passed filtered fluorescence having a wavelength of between 430 nm and 560 nm and a red filter type configured to preferentially pass filtered fluorescence having a wavelength of 560 nm or more. Of course, it will be appreciated that any suitable number of types of filter may be used. For example, the filter arrangement 21 may comprise a mosaic formed of two types of optical filters and two polarisation filters having orthogonal polarisations. In FIG. 2, the two filter types F(a), F(b) are shown separated for clarity, but it will be appreciated that the filter arrangement may form a single layer mosaic of the filter types. In particular, the mosaiced filter may preferably be positioned directly in front of the image sensor 31, such that a first subset of pixels P(a) receive light passed through a first filter type F(a) and a second subset of pixels P(b) receive light passed through a second filter type F(b). Of course, if more filter types are used, then the image sensor is divided into a larger number of pixel subsets, which each receive light passed through a single filler type. In some embodiments, the filter arrangement takes the form of a Bayer filter, for example having red, green and blue optical filter types. By providing a filter arrangement 21 and image sensor 31 in this way, data may be read out from pixel subsets P(a), P(b) of the image sensor 31 (each pixel subset P(a), P(b) of the image sensor 31 may relate to a ‘channel’ of data), and the data from each pixel subset P(a), P(b) compared in order to identify the dentine-enamel boundary in the oral region of interest. For example, a ratiometric image may be formed by taking a ratio of adjacent pixels in different subsets.

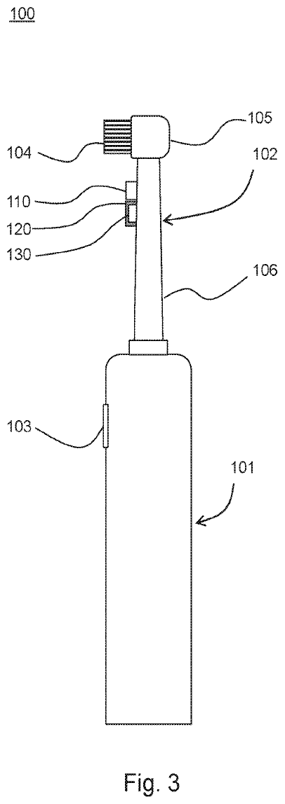

Dentine-enamel detection systems according to the present invention may be implemented in oral treatment devices. FIG. 3 is a schematic drawing of an oral treatment device according to the present invention. The oral treatment device in the present instance is a toothbrush, although in other embodiments the oral treatment device may be a flossing device, an oral irrigator, an interproximal cleaning device, an oral care monitoring device, or any combination of such.

In this embodiment, the oral treatment device 100 comprises a handle 101 and a tool head 102. The handle 101 forms the main body of the device 100 and may be gripped by a user during use of the device 100. It is generally cylindrical in shape. In the embodiment shown in FIG. 3, the handle 101 comprises a user interface 103. The user interface 103 comprises a user operable button configured to be depressible by the user when the user is holding the handle. This may be operable to control e.g. a power state of the oral treatment device 100, or to provide user input to the oral treatment device in a known manner.

The tool head 102 comprises a plurality of bristles 104 for performing a tooth brushing function, although as discussed above, an oral treatment device according to the present invention may take other forms not requiring the presence of bristles. For example, in some other embodiments the oral treatment device 100 comprises a dedicated fluid delivery device, e.g. for performing a treatment such as cleaning gaps between adjacent teeth, and/or for delivering a cleaning or whitening medium to the teeth of the user. The tool head 102 comprises a head portion 105 and a stem portion 106. The stem portion connects the handle to the head portion of the tool head 102 The stem portion 106 is elongate in shape, which serves to space the head portion of the tool head from the handle to facilitate user operability of the oral treatment device 100. The head 102 and/or the stem 106 may be detachable from the handle 101.

As mentioned above, in this embodiment, the dentine-enamel boundary detection system is incorporated in the oral treatment device. Many components of the boundary detection system are not shown in this figure. However, a light emission module 110 in the form of an LED is provided on the stem portion 106 of the tool head 102. A sensor module 130 is also provided on the stem portion 106 of the tool head and is covered by an optical filter arrangement 120, so that the optical filter arrangement 120 is arranged in an optical path between the oral region of interest and the sensor module 130. The advantage of providing the light emission module 110 and/or the sensor module 130 on the tool head 102 is that it allows for improved intraoral imaging.

Whilst in this embodiment the light emission module 110 and the sensor module 130 are provided on the tool head 102, it is also contemplated than in some embodiments, these components may be located in the handle/body 101 of the device, and one or more guide components may be used for receiving and delivering light to and from the light emission module 110 and the sensor module 130. For example, the device may comprise one or more guide channels extending from an aperture provided on the tool head 102, along (e.g. within) the stem portion 106, to the light emission module 110 and the sensor module 130 located in the handle of the device. In such an arrangement the guide channel may comprise one or more fibre optic cables.

It will be understood that the oral treatment device 100 may further comprise additional components that are not shown or described here, e.g. a power source such as a battery, or other components conventionally present in oral treatment devices such as electric toothbrushes, flossing devices, oral irrigators, interproximal cleaning devices. or oral care monitoring devices.

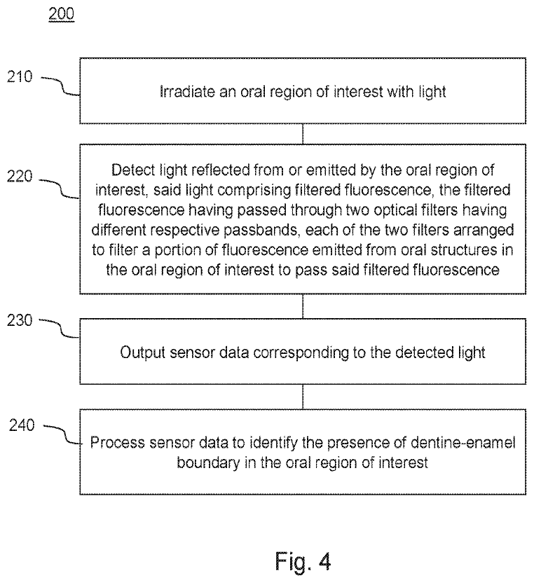

FIG. 4. is a flow diagram showing a method 20 of operating a boundary detection system, or oral treatment device incorporating a boundary detection system according to the present invention. The method 200 may be used to operate the dentine-enamel boundary detection system 1 or oral treatment device 100 described above with reference to FIG. 1 and FIG. 2. The method 200 is performed at least in part by the processor module 40a of the boundary detection system.

In step 210, a light emission module 10, 110 is used to irradiate an oral region of interest with light. In line with the discussion of FIG. 1, in this embodiment the light is laser light having a wavelength of 405 nm, although other wavelengths may be used.

In step 220, the sensor module 30. 130 detect slight reflected from or emitted by the oral region of interest, said light comprising filtered fluorescence, the filtered fluorescence having passed through two optical filters (e.g. in a filter arrangement 20. 120) having different respective passbands, each of the two filters arranged to filter a portion of fluorescence emitted from oral structures in the oral region of interest to pass said filtered fluorescence.

In step 230, the sensor module 30, 130 outputs sensor data (in this embodiment, generated image data representing at least a portion of the oral cavity of the user).

In step 240, the generated image data is processed to identify the presence of dentine-enamel boundary in the oral region of interest. In some embodiments, the data may be further processed to determine location data indicating a location of an interproximal gap between adjacent teeth in the oral cavity of the user. For example, the generated image data is processed using a trained classification algorithm configured to identify interproximal gaps. The trained classification algorithm may be trained prior to the use of the oral treatment device.

When the method is used for control of an oral treatment device, the method may include a further optional step (not shown) of controlling the oral treatment device 100 to deliver a treatment to a detected interdental gap.

By use of this method, a dentine-enamel boundary can be detected automatically, through use of the sensor module and trained classification algorithm, and in a more accurate manner than may be possible using existing systems. Furthermore. in systems or devices configured for delivery of one or more treatments within an oral cavity (for example, in systems configured for jetting of fluid at or adjacent to identified oral structures), treatment delivery can be controlled accordingly, without the need for user input. This can have significant advantages. For example, the user does not need to determine when an oral treatment device incorporating the boundary detection system is in a position that is suitable for treatment delivery. Instead. such a determination can be made automatically based on intraoral image data generated by the sensor module and can be performed in substantially real-time.

The method may be implemented at least in part by computer software stored in (non-transitory) memory and executable by the processor, or by hardware, or by a combination of tangibly stored software and hardware (and tangibly stored firmware). Embodiments also extend to computer programs. particularly computer programs on or in a carrier, adapted for putting the above-described embodiments into practice. The program may be in the form of non-transitory source code, object code, or in any other non-transitory form suitable for use in the implementation of processes according to embodiments. The carrier may be any entity or device capable of carrying the program, such as a RAM, a ROM, or an optical memory device. etc.

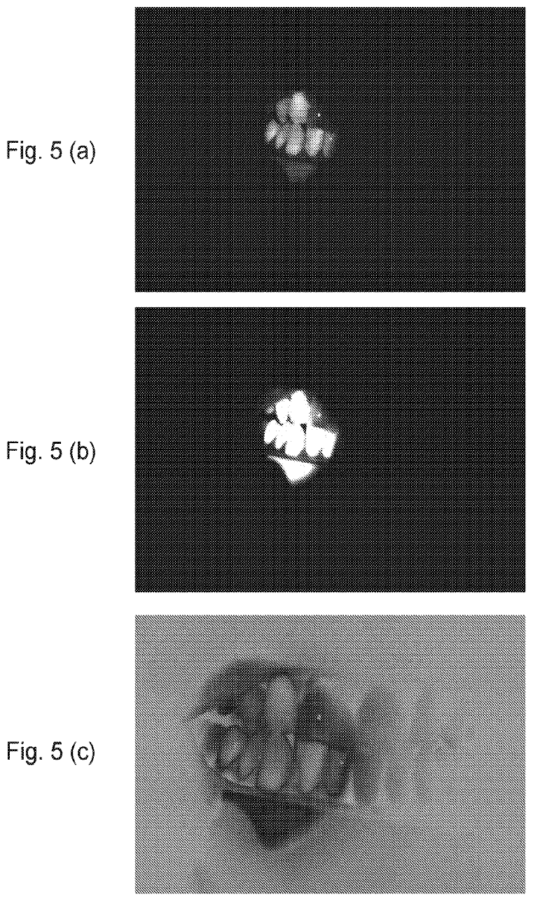

FIG. 5 shows an example of data from two channels of an imaging device, such as the filter arrangement 21 and the image sensor 31 described above with respect to FIG. 2, and a ratiometric image derived from the two channels. In particular, FIG. 5 is an example using optical filters, but it will be appreciated that a similar result would be achieved if the filter arrangement and/or channels used related to different polarisation states (e.g. orthogonal polarisations).

FIG. 5(a) shows a first channel of an image of oral structures. In particular, FIG. 5(a) shows an image derived from a first subset of pixels of an image sensor, wherein the first subset of pixels were exposed to fluorescence received from an oral region of interest after passing through a red filter configured to preferentially pass filtered fluorescence having a wavelength of 560 nm or more.

FIG. 5(b) shows a second channel of an image of oral structures. In particular, FIG. 5(b) shows an image derived from a second subset of pixels of an image sensor, wherein the second subset of pixels were exposed to fluorescence received from an oral region of interest after passing through a blue filter configured to preferentially pass filtered fluorescence having a wavelength of between 430 nm and 560 nm.

FIG. 5(c) shows a ratiometric image which has been derived from FIGS. 5(a) and 5(b). In particular, FIG. 5(c) was obtained by taking the ratio of the intensities at each pixel position in FIGS. 5(a) and 5(b) and displaying the result. It will be appreciated that FIG. 5(c) shows a dark band around the periphery of each tooth in the image, which highlights the dentine-enamel boundary at a region of each tooth where enamel erosion principally occurs, exposing the underlying dentine.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A dentine-enamel boundary detection system for an oral inspection device, the dentine-enamel boundary detection system comprising:
a light emission module configured to emit light to irradiate an oral region of interest;
two optical filters having different respective passbands, each of the two optical filters arranged to filter a portion of fluorescence emitted from oral structures in the oral region of interest to pass filtered fluorescence;
a sensor module configured to detect the filtered fluorescence and output corresponding sensor data; and
a processor module configured to identify the presence of a dentine-enamel boundary in the oral region of interest by processing the sensor data output by the sensor module.

2. The dentine-enamel boundary detection system according to claim 1, wherein the light emission module is configured to emit light having a wavelength in a range of 405 to 450 nm.

3. (canceled)

4. The dentine-enamel boundary detection system according to claim 1, wherein the two optical filters include a blue filter configured to preferentially pass filtered fluorescence having a wavelength of between 430 nm and 560 nm and a red filter configured to preferentially pass filtered fluorescence having a wavelength of 560 nm or more.

5. The dentine-enamel boundary detection system according to claim 1, wherein:
the sensor module is configured to detect a time of arrival at the sensor module of the filtered fluorescence and output corresponding time data, and
the processor module is further configured to process the time data output by the sensor module to identify the presence of a dentine-enamel boundary in the oral region of interest.

6. The dentine-enamel boundary detection system according to claim 5, wherein the sensor module comprises a single photon avalanche photodiode.

7. The dentine-enamel boundary detection system according to claim 1, further comprising:
two polarisation filters having orthogonal polarisations, each of the two polarisation filters arranged to filter a portion of the fluorescence emitted from oral structures in the oral region of interest to pass polarised fluorescence.

8. (canceled)

9. The dentine-enamel boundary detection system according to claim 1, wherein the processor module is configured to identify the presence of an interdental gap in the oral region of interest.

10. (canceled)

11. An oral inspection or treatment device comprising:
a dentine-enamel boundary detection system comprising:
a light emission module configured to emit light to irradiate an oral region of interest,
two optical filters having different respective passbands, each of the two optical filters arranged to filter a portion of fluorescence emitted from oral structures in the oral region of interest to pass filtered fluorescence,
a sensor module configured to detect the filtered fluorescence and output corresponding sensor data, and
a processor module configured to identify the presence of a dentine-enamel boundary in the oral region of interest by processing the sensor data output by the sensor module;
a body; and
a cleaning tool head operably coupled to the body.

12. The oral inspection or treatment device according to claim 11, wherein one or both of the light emission module and the sensor module of the dentine-enamel boundary detection system are provided on the cleaning tool head.

13. (canceled)

14. The oral inspection or treatment device according to claim 11, wherein the processor module is provided as part of a controller module located within a remote device, wherein the remote device and the oral inspection or treatment device are configured for communication with one another to allow for exchange of signals and/or data.

15. The oral inspection or treatment device according to claim 11, wherein, on identification of the presence of an interdental gap in the oral region of interest, the oral inspection or treatment device is configured to apply a treatment to the identified interdental gap in the oral region of interest.

16. A method for detecting a dentine-enamel boundary in an oral region of interest, comprising steps of:
irradiating an oral region of interest with light;
detecting light reflected from or emitted by the oral region of interest, said light comprising filtered fluorescence, the filtered fluorescence having passed through two optical filters having different respective passbands, each of the two optical filters arranged to filter a portion of fluorescence emitted from oral structures in the oral region of interest to pass said filtered fluorescence;
outputting sensor data corresponding to the detected filtered fluorescence; and
processing said sensor data to identify the presence of a dentine-enamel boundary in the oral region of interest based on the filtered fluorescence.

17. The method for detecting a dentine-enamel boundary according to claim 16, wherein processing said sensor data to identify the presence of a dentine-enamel boundary in the oral region of interest comprises creating a ratiometric image of the oral region of interest based on the ratio of intensity of filtered fluorescence passed through the two optical filters.

18. The method for detecting a dentine-enamel boundary according to claim 16, wherein the light irradiating the oral region of interest has a wavelength in a range of 405 to 450 nm.

19. The method for detecting a dentine-enamel boundary according to claim 16, wherein:
outputting sensor data corresponding to the detected light comprises outputting time data identifying the time of arrival at which said light is detected, and
processing said sensor data to identify the presence of a dentine-enamel boundary in the oral region of interest comprises processing the time data.

20. The method for detecting a dentine-enamel boundary according to claim 19, wherein processing said sensor data to identify the presence of a dentine-enamel boundary in the oral region of interest comprises creating a fluorescence lifetime image based on the time data.

21. The method for detecting a dentine-enamel boundary according to claim 16, wherein detecting light reflected from or emitted by the oral region of interest comprises detecting polarised fluorescence, the polarised fluorescence having passed through two polarisation filters having orthogonal polarisations, each of the two polarisation filters arranged to filter a portion of fluorescence emitted from oral structures in the oral region of interest to pass said polarised fluorescence.

22. The method for detecting a dentine-enamel boundary according to claim 21, wherein processing said sensor data to identify the presence of a dentine-enamel boundary in the oral region of interest comprises calculating the Stokes parameters from the orthogonal polarization directions of the polarised fluorescence.

23. The method for detecting a dentine-enamel boundary according to claim 21, wherein processing said sensor data to identify the presence of a dentine-enamel boundary in the oral region of interest comprises creating a ratiometric image of the oral region of interest based on the ratio of intensity of polarised fluorescence passed through the two polarisation filters.

24. The method for detecting a dentine-enamel boundary according to claim 16, wherein irradiating an oral region of interest with light comprises irradiating an oral region of interest with polarised light.

25. (canceled)
